(19) United States
(12) Patent Application Publication
(10) Pub. No.: US 2003/0050538 A1
(43) Pub. Date: Mar. 13, 2003

(54) System and method for medical observation system located away from a hospital

(76) Inventors: Morteza Naghavi, Houston, TX (US); Mohammad Madjid, Houston, TX (US); Parsa Mirhaji, Houston, TX (US); Reza Mohammadi, New York, NY (US); David J. Robinson, Houston, TX (US)

Correspondence Address: Gary R. Maze, Duane Morris LLP, One Greenway Plaza, Suite 500, Houston, TX, 77046, US

(21) Appl. No.: 10/157,208

(22) Filed: May 29, 2002

Related U.S. Application Data
(60) Provisional application No. 60/294,040, filed on May 29, 2001.
(60) Provisional application No. 60/293,965, filed on May 29, 2001.
(60) Provisional application No. 60/293,964, filed on May 29, 2001.
(60) Provisional application No. 60/293,897, filed on May 29, 2001.

(51) Int. Cl. A61B 5/00

(52) U.S. Cl. 600/300

(57) ABSTRACT

The present invention comprises a system and method for a home diagnostic observation center useful for early risk assessment of a patient experiencing chest discomfort associated with a potentially emergency heart condition. The system comprises a computer comprising a persistent data store, an input device, and an output device; a data network operatively in communication with the computer; a heartfolio database comprising an individualized, heart health portfolio for the patient; an interactive questionnaire, displayable on the output device, which can interactively accept input from the patient relating to the patient's current symptoms and clinical information and store the input in the heartfolio database; a medical apparatus, operatively in communication with the computer, which can acquire predetermined physiological data of the patient; and medical diagnostic software executing at least partially in the computer. It is emphasized that this abstract is provided to comply with the rules requiring an abstract which will allow a searcher or other reader to quickly ascertain the subject matter of the technical disclosure. It is submitted with the understanding that it will not be used to interpret or limit the scope of meaning of the claims.

### RELATED APPLICATIONS

The present invention claims priority from U.S. Provisional Application Ser. No. 60/294,040 filed May 29, 2001, U.S. Provisional Application Ser. No. 60/293,965 filed May 29, 2001, U.S. Provisional Application Ser. No. 60/293,964 filed May 29, 2001, and U.S. Provisional Application Ser. No. 60/293,897 filed May 29, 2001.

### BACKGROUND OF THE INVENTION

The present invention relates to out-of-hospital, early risk assessments of patients experiencing physiological symptoms such as chest discomfort that might be possibly related to an emergency heart condition.

Evaluation of acute chest pain remains challenging, despite many insights and innovations over the past two decades. Heart attack has remained as a the number one killer of the adult population in the United States. Every 29 seconds, one American suffers from an unexpected heart attack. More than 225,000 heart attack victims die before reaching the hospital.

Innovations in reperfusion therapies can potentially reduce CHD morbidity and mortality associated with acute myocardial infarction (AMI) when treatment is initiated within the first few hours of symptom onset. Unfortunately delayed access to medical care in patients with acute myocardial infarction is unbelievably common and has lead to our failure in reducing out-of-hospital heart mortality of heart attacks.

More than 60% of patients delay more than 3 hours before deciding to go to hospital and less than 20% of patient is receive on-time life saving cardiac therapy due to these delays. Although fifty percent of patients with heart attacks have a history of stuttering chest symptoms before they crash most heart attack patients choose a “wait and see” approach, hoping their symptoms would lessen or disappear. As a result, too many people who experience heart attacks waste their critical time to prevent further damage to their heart. Since there is not an easy way for them to check their condition or consult with a medical professional, they waste their time hoping their conditions become better. The unacceptably lengthy delay results in most patients 1 make them ineligible for advanced medicine therapies such as pharmacological reperfusion strategies.

A system which could signal early high-risk cardiac disease at a location physically removed from a hospital, e.g. a home or other residence, has not yet been proposed, and previous attempts such as public campaigns provided education to general population proved inefficient and largely unsuccessful. Most researchers now believe that early strategy that could risk stratify heart disease out of hospital may be more effective than public awareness program.

There are many reasons as to why present heart attack treatment strategy should change. The major reasons include: for those with suspected high risk cardiac disease, public awareness strategies do not reduce access times for patient 1s with chest pain; the health care system involvement in heart attack starts too late; heart attacks begin in community, but physicians are unable to intervene until patient 1 accesses the emergency medical systems or comes to the emergency departments.

A system for early out-of-hospital detection and risk stratification would likely decrease patients' indecision when high risk disease is identified.

Another problem with present systems for managing heart disease occurs for the low risk population.

A significant number of unnecessary emergency visits for chest pains related to other non-urgent non-cardiac conditions. Almost 70% of over 5 million people annually in the U.S., who visit the ER with a complaint of chest pain, are not having a heart attack, but are diagnosed with non-cardiac or non-specific chest pain. This number is an average annual rate of 27.7 visit visits per 1,000 patient 1s. Chest pain is the second reason for visiting the ER after stomach pain and even more than fever. This disturbing statistic places a huge financial burden on the health care industry. The cost for hospital-based, non-specific chest pain is anywhere from $1,000 to $15,000 per patient. In 1994, there were 10.9 million visits to the ER for ECG service, including: an estimated 4.6 million annual emergency department (ED) visits with complaints of non-traumatic chest pain, translating into an average annual rate of 27.7 visits per 1,000 persons; between 40-50% of the ER visits are not urgent; 70% of patient Is in critical care units turn out not to have myocardial infarction; and the average hospital costs for such patients are $5,000-$6000.

Specialized hospital-based chest pain units (observation centers) have been developed to provide a comprehensive triage system in the Emergency Department that is cost effective for patients who present with various types of chest discomfort. A number of protocols and systems have been designed to risk stratify chest pain patients in hospitals. This system allows effective triage of high risk presentations into the hospital intensive care units while allowing the physicians to safely discharge the low immediate cardiac risk presentations. The present invention is modeled after ED methods of dynamic risk stratification and management.

The present invention combines a computer such as a personal computer with a data communications interface such as to the Internet and one or more low-cost medical devices to provide a new strategy addressing medical delays for high risk cardiac disease when those with high risk cardiac disease are not in a hospital setting while reducing unnecessary ED visits.

As an online chest pain center deployable in a wide variety of environments located away from a clinical environment such as a hospital, e.g. a home or other residence, a method of the present invention employs a computer devices, e.g. personal computers including laptops, handheld devices such as personal digital assistants, tablet PCs, and the like, combined with a specially designed interactive questionnaire 40 useful in obtaining data regarding a patient's current symptoms and past medical record. The present invention may allow patient access using numerous communication devices, e.g. Internet browsers, cell phones, instant messaging systems, and the like, and employs an apparatus for obtaining physiological data (ECG, BP, etc) from patients, and transfers information through any media (internet, LAN, telephone lines, cell-phone network, Cable TV network, etc). The present invention further comprises a specialized intelligent, trainable, integrated data base software whose design is based on a predetermined set of observed and/or predicted responses to a given set of symptoms as would be made by and/or gleaned from a trained medical practitioner, herein sometimes referred to as “Physician Cognitive Engineering”. Physician Cognitive Engineering may include specialized imitations of specialty medical practitioners, e.g. modeling of cardiologists may be described as Cardiology Cognitive Engineering.

In a preferred embodiment, the invention introduces an easily accessible, out-of-hospital, tele-triage, decision support and risk stratification system to the public domain. It provides early management to out-of-hospital users based on both their background medical conditions and real time clinical and physiological data.

### BRIEF DESCRIPTION OF THE DRAWINGS

FIG. 1 is a schematic diagram of a system according to the present invention;

FIG. 2 is a schematic diagram of a system according to the present invention in a deployed embodiment;

FIG. 3 is a schematic overview of a specialized operating system of the present invention;

FIG. 4 is a schematic representation of a modeling method of the present invention involving physician cognitive engineering;

FIG. 5 is schematic representation of a modeling method of the present invention involving risk stratification;

FIG. 6 is an exemplary presentation of an exemplary heartfolio;

FIG. 7 is an exemplary presentation of an exemplary interaction questionnaire;

FIG. 8 is a plan view in partial representative of an exemplary medical device and sensor;

FIG. 9 is a plan view in partial representative of an exemplary integrating device;

FIG. 10 is a plan view in partial representative of a second exemplary integrating device;

FIG. 11 is a diagrammatic representation of a method of use of the present invention; and

FIG. 12 is a diagrammatic representation of a method of use of the present invention.

### DETAILED DESCRIPTION OF AN EXEMPLARY EMBODIMENT

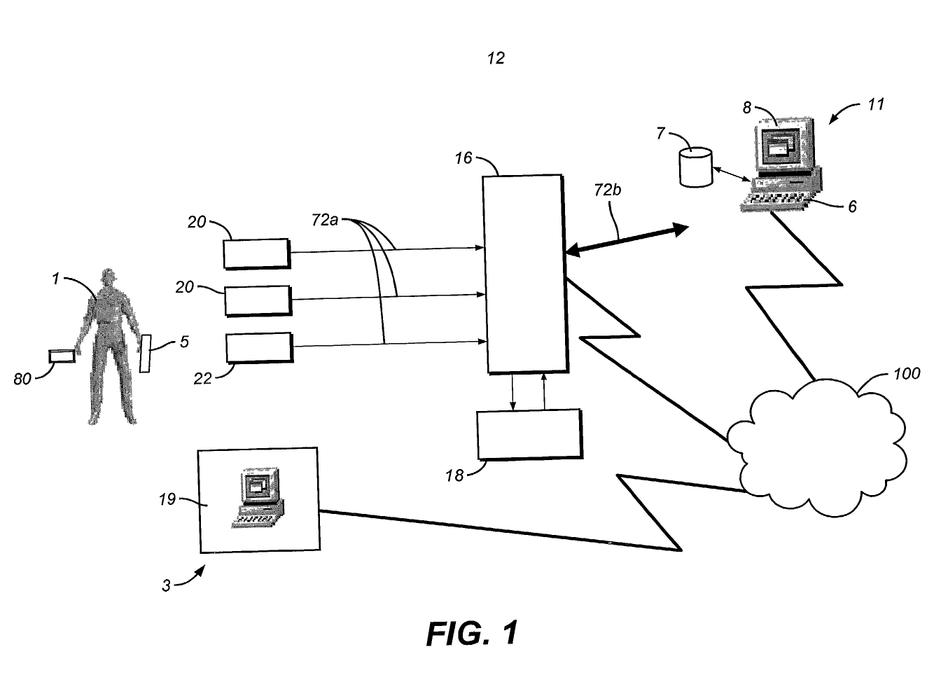

Referring now to FIG. 1, a generalized schematic of an exemplary embodiment, the present invention, generally referred to herein as personal healthcare management system 10, comprises components that work together to provide an out-of-hospital, affordable, real time, automated intelligent system for early managing and risk stratifying of patients 1 based on their symptoms, physiologic data (ECG, BP) and their past medical history. As used herein, “home” and “home-based” are understood to broadly mean locations that are not hospitals or other clinical facilities. Further, although the exemplary embodiment is described in terms of cardiac care and heart related symptoms and measurements, the present invention is not limited to cardiac care.

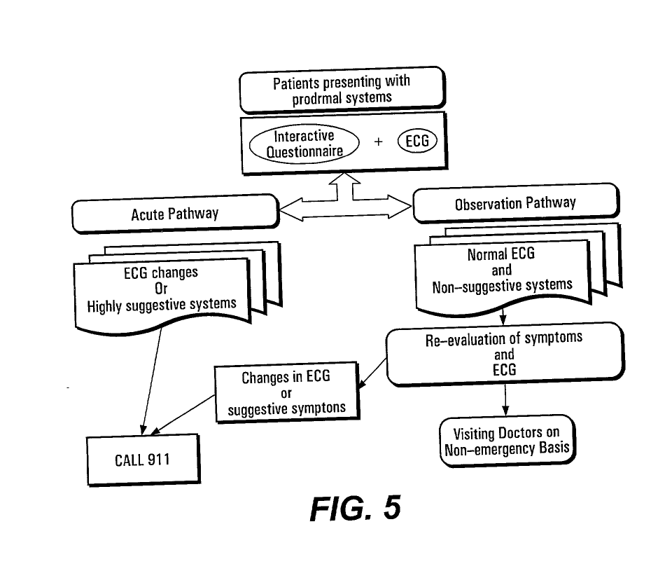

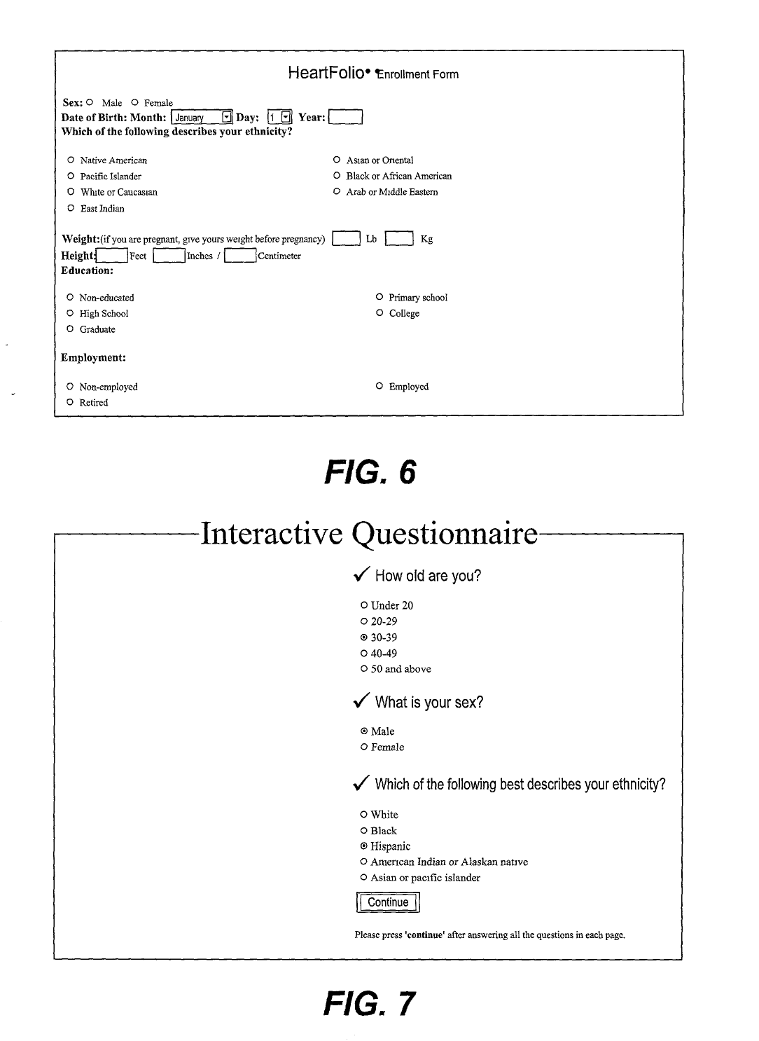

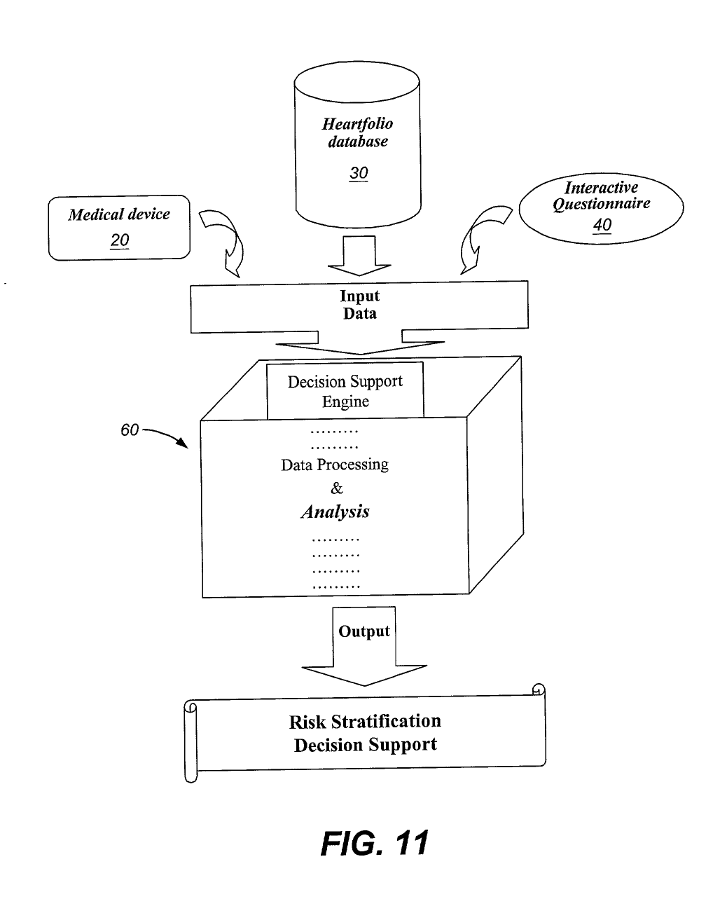

Personal healthcare management system 10 comprises computer 11 comprising input device 6, e.g. a mouse, keyboard, biometric input, microphone, and the like; persistent data store 7 such as fixed or removable magnetic, electronic, and/or optical media, and output device 8 such as a monitor. As used herein, computer 11 may comprise a personal computer, a personal digital assistant, a handheld computer, a laptop computer, or the like. Personal healthcare management system 10 further comprises healthfolio 31 (FIG. 6), heartfolio database 30 (FIG. 11), interactive questionnaire 40 (FIG. 3), medical device 20, specialized healthcare operating system 50 (FIG. 5), and medical diagnostic software 60.

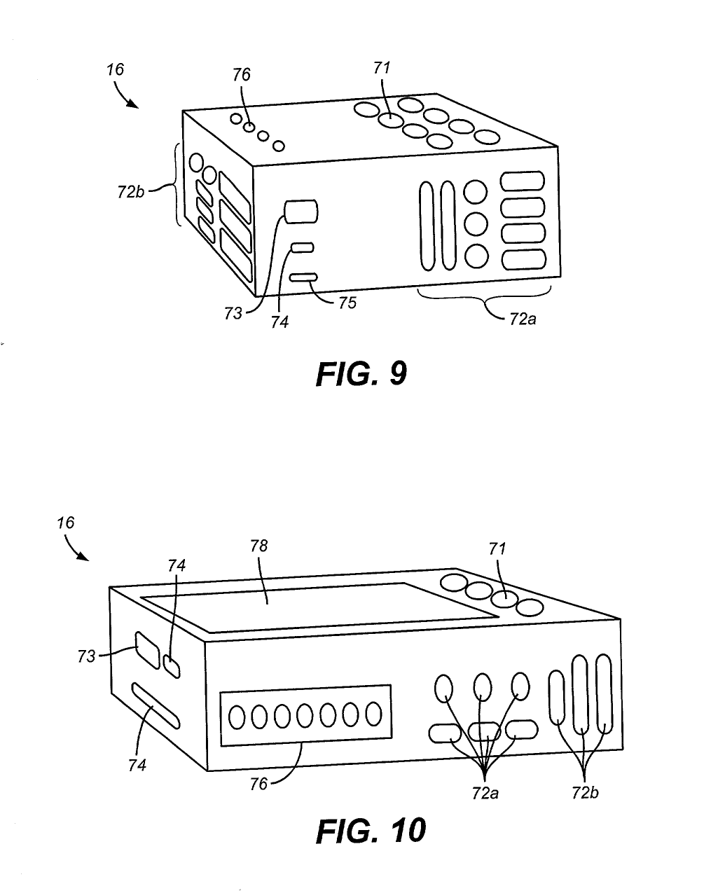

Personal healthcare management system 10 comprises computer 11 operatively in communication with one or more home-based medical devices 20 including sensors 22. Personal healthcare management system 10 may also provide a full-featured personal computer environment in which third party applications may be installed and invoked. Specialized healthcare specific operating system 50 operates on personal healthcare management system 10. Additionally, personal healthcare management system 10 may comprise communication link 12 (not shown in the figures), expansion port 14 (not shown in the figures), and integrating device 16 (FIG. 9 and FIG. 10). Interactive user control module 5 (FIG. 1) and programmable remote paging device 80 (FIG. 1) may also be provided.

Computer 11 provides standard personal computer functionality and may be implemented using standard personal computer technology, e.g. input device 6 such as a keyboard, output device 8 such as video display, a CPU, memory such as RAM memory, and persistent data store 7 such as a fixed or removable electronic, magnetic, and/or optical medium.

Personal healthcare management system 10 may further comprise communication link 12 to allow data communication over public or private data networks 100, e.g. the Internet, local area networks, telephone or cellular phone network, cable systems, and the like, or combinations thereof.

Personal healthcare management system 10 can automatically recognize and simultaneously control and acquire data from a plurality of medical devices 20. Medical devices 20 may be operatively in communication with personal healthcare management system 10 via wired or wireless communication links, e.g. serial, parallel, USB, infrared, flashcard, PCMCIA, SCSI, BlueTooth®, or IEEE 1394 ports, or the like, or combinations thereof.

In addition, medical devices 20 and/or sensors 22 may be attached to personal healthcare management system 10 via a specially designed expansion-hosting device such as an external integrating device 16. Additionally, expansion-hosting device 16 may comprise expansion port 14 (not shown in the figures) that can host assembly housing 24 (not shown in the figures) comprising a plurality of medical devices 20,22 and/or specially designed integrating device 16 connected to a plurality of medical devices 20,22. For example a digital pregnancy tester, a glucometer and a digital urine analyzer can be packed together into assembly housing 24 and attached to personal healthcare management system 10 through expansion port 14.

Other manufacturers may use an internal version of integrating device 16 and internal versions of medical devices 20 assembled together into a single housing. For example, to turn a notebook computer system to personal healthcare management system 10, a PCMCIA version of integrating device 16 can be used with a notebook computer to integrate medical devices 20 and help turn the notebook computer into personal healthcare management system 10.

Medical devices 20 may be internal or external to personal healthcare management system 10 and may comprise an electrocardiograph device, a blood pressure measurement device, thermometers, digital biochemical devices, glucometers, and physiological laboratory kits, digital biomechanical monitoring devices, peak-flow meter, and the like, or combinations thereof. Additionally, medical devices 20 may be external or internal to personal healthcare management system 10.

A variety of sensors 22 for obtaining biophysical, biomechanical, physiological, biochemical, and electromechanical parameters, e.g. ECH electrodes, may be used to connect patients to personal healthcare management system 10 through a wired or wireless communication links, e.g. serial, parallel, USB, infrared, or IEEE 1394 ports, or the like, or combinations thereof. For example, a wireless bra may be used where the wireless bra comprises one or more sensors 22 which transmit data obtained from patient 1, e.g. from an ECG electrode, a digital stethoscope, and a digital thermometer integrated inside the wireless bra.

Additionally, processor 18 may be used to interface with computer 11 and be coupled with integrating device 16 acting as a signal conditioner module to aid in integrating medical devices 20 with computer 11. Integrating device 16 may be used as a signal conditioner module to condition data inputs when coupling medical devices 20 into computer 11 and/or processor 18.

Personal healthcare management system 10 utilizes interactive user control module 5 (FIG. 1) to facilitate interaction between patient 1 and personal healthcare management system 10. User control module 5 (FIG. 1) may be accessed by patient 1 using any of numerous functionally equivalent input devices, e.g. a touch screen, a touch pad, speech synthesis via a microphone, keyboards, mice, recognition devices, remote controls, biometric devices, and the like or combinations thereof. Interactive user control module 5 (FIG. 1) may be implemented as a device an externally attachable to personal healthcare management system 10 or can be built in the same physical system assembly of personal healthcare management system 10. Moreover, personal healthcare management system 10 may possess more than one embodiment of interactive user control module 30. For example, interactive user control module 5 (FIG. 1) can use a built-in touch screen and a remote control.

Interactive user control module 5 (FIG. 1) may be further tailored for a specific healthcare condition of patient 1 and/or configuration of personal healthcare management system 10. For example, interactive user control module 5 (FIG. 1) may be specially designed as a touch screen for an elderly patient 1, as a speech sensitive device for a debilitated patient 1, or integrated into specially designed portion of furniture such as a sliding side panel for personal computer integrated into a bed or couch.

Additionally, interactive user control module 5 (FIG. 1) may be used to facilitate access to one or more predefined functions of personal healthcare management system 10. For example, interactive user control module 5 (FIG. 1) may comprise a remote control device (not shown in the figures) with one or more dedicated buttons or keys. These dedicated keys or buttons may be assigned to a specific function or group of functions, e.g. instant 911 dialer, instant ECG recording, instant medical record preview, or the like.

By way of further example, a couch employing the present invention may comprise joints and gears sufficient to further extend the couch into a bed, allowing patient 1 to lie on the extended couch comfortably while personal healthcare management system 10 is running ECG monitoring. Sensory devices 22, display 8, and interactive user control module 5 (FIG. 1) may then be designed and built into the couch, e.g. in a automatically launched, hidden box within the couch. Patient 1 can also use personal healthcare management system 10 as a healthcare management device or as an ordinary PC, comfortably while sitting on the couch.

Additionally, access to personal healthcare management system 10 may be accomplished using programmable remote paging device 80 (not shown in the figures). This allows patient 1 to be connected to and/or monitored by personal healthcare management system 10 when patient 1 is not located proximate personal healthcare management system 10. Programmable remote paging device 80 may be implemented in a plurality of designs and forms according to the comfort and health condition requirements of patient 1. For example, programmable remote paging device 80 may be a conventional paging device, an eyeglass or earring-like paging device comprising a speaker or beeper, a watch-like device comprising vibrator, and the like, or combinations thereof.

Personal healthcare management system 10 may be used to support a variety of automated paging strategies which may be employed to automatically send or route notifications to or from patient 1 or another, e.g. a physician, using these paging strategies. For example, personal healthcare management system 10 may be configured to automatically call a predefined phone number to notify a designated person or entity about a healthcare event. Programmable paging devices 40 may perform a plurality of different alerting actions, e.g. blink, beep, talk through a speech synthesis device, vibrate, and the like, These may be invoked according to a pre-defined set of configurable criteria.

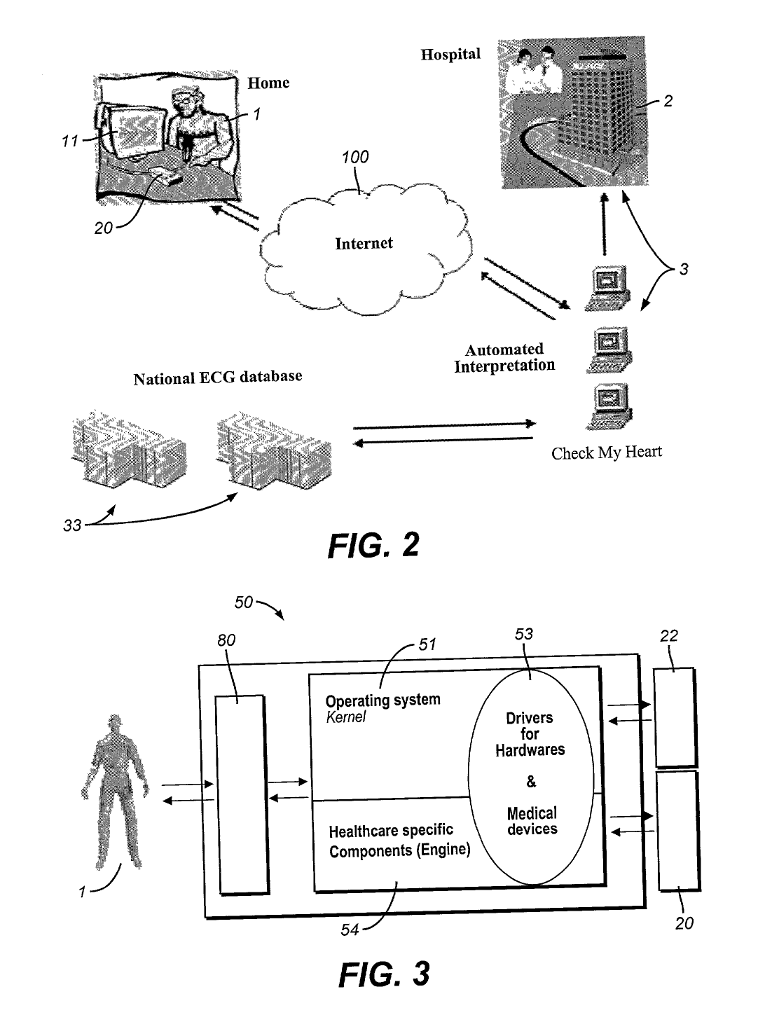

Referring now to FIG. 2, personal healthcare management system 10 gathers necessary information of patient 1 such as past medical history, symptoms, risk factors, ECGs, and the like, and stores them in heartfolio database 30. Specialized healthcare operating system 50 may submit required health data acquired from different sources and applications or by patient 1 directly into the compilation of healthfolio 31. Health data acquired by a certain medical device 20 in a certain application environment may be accessible to other healthcare applications or for communication through data network 100.

In a preferred embodiment, heartfolio database 30 is a secure, HIPAA (Health Insurance Portability and Accountability Act) compliant database. Personal healthcare management system 10 uses the stored information to create a updateable personalized portfolio 31, e.g. a cardiac portfolio 31, for each patient 1. Personal healthcare management system 10 may be accessible through data network 100, e.g. the Internet, where such access may be via dialup, DSL, satellite, or other data communications to a clinical location such as hospital 2 or a service which can provide most if not all of the functionality of personal healthcare management system 10 to patient 1 when personal healthcare management system 10 is not otherwise available to patient 1, e.g. remote service 3. Personal healthcare management system 10 processes the current symptoms, past medical clinical information 30 of patient 1 and dynamic physiologic data for risk stratification and management of patient 1 and creates an output based on the all available information for patient 1. Generated results may include real time risk stratification and automated emergency, e.g. 911, activation, or transmitted pertinent cardiac information to hospitals or health care providers, or other interested parties.

Personal healthcare management system 10 is customizable, containing baseline data not available to hospitals since patient 1 can store baseline data prior to the onset of an acute event. This output ranges from simple patient advice to more sophisticated actions like activating an alarm system or informing health care provider automatically. The invention can provide therapeutic actions such as perform cardioversion as an automated external defibrillator.

Additionally, personal healthcare management system 10 may have access to alternative databases such as a national ECG database 33.

Referring now to FIG. 3, specialized healthcare specific operating system 50 comprises one or more drivers and software components as well as a specially designed healthcare specific graphical user interface to automatically recognize, and work with, medical devices 20,22. Additionally, specialized healthcare specific operating system 50 presents an interface 80 to aid in guiding patient 1 through predetermined functions of specialized healthcare specific operating system 50.

Specialized healthcare operating system 50 may be installed and used in standard computers 11 (FIG. 1), e.g. desktop computers, laptop and notebook computers, handheld computers, and the like. In a preferred embodiment, in addition to incorporating basic input/output system routines 51 and utilities found in certain existing personal computer operating systems, specialized healthcare operating system 50 possesses a comprehensive collection of drivers and software components 53 to automatically recognize, control, and work with a variety of home based medical devices 20 attached to personal computer 11 and manage health data.

Drivers 53 support a wide variety of medical devices 20 and enable specialized healthcare operating system 50 to efficiently connect and interact with medical devices 20. Specialized healthcare operating system 50 can include a specific driver 53 for each medical device 20 or can include general drivers for divers types of medical devices 20.

Specialized healthcare operating system 50 contains a collection of re-usable and shared components, application programming interfaces (API) and applications based on the medical and healthcare standards and protocols, generally referred to as “54” in FIG. 3. Application programming interfaces and shared components are publicly accessible for programmers and system developers. APIs and shared components may be based on standard definitions such as offered by authorized organizations like ANSII, IEEE, American Society Of Testing And Materials (ASTM), Health Informatics Standards Planning Panel (HISPP), Message Standards Developers Subcommittee (MSDS) and the like. Accordingly, support for standards like X12, HL7, DICOMM, and MLM may be provided. Medical standards supported by specialized healthcare operating system 50 help assure standardization, security, interoperability and efficient data exchange between diverse healthcare applications and medical devices 20 built on this platform and create a rich software platform for developing new generation of robust healthcare management applications along with a more comprehensive health folio.

Specialized healthcare operating system 50 can also provide a full-featured conventional operating system environment to access ordinary functions of an ordinary PC through health-specific GUI 80 (read, write, execute, etc). In an embodiment, specialized healthcare operating system 50 comprises a full-featured operating system with multimedia support, networking support, multi-user environment, security features, multi-tasking and multithreading capabilities. Specialized healthcare operating system 50 may therefore be used as a platform for a variety of software applications already developed for conventional operating system environments.

In an embodiment, specialized healthcare operating system 50 is exclusively designed and developed as a health-care specific operating system. All drivers 53, software components 54, and GUI 80 are designed and developed exclusively for specialized healthcare operating system 50. In a further embodiment, specialized healthcare operating system 50 is a healthcare specific operating system adapted from an existing operating system platform, e.g. Microsoft® Windows®, Linux, Apple® Macintosh®, and the like. In these embodiments, the hosting operating system and GUI 80 are rebuilt and additional components are added. All of these are then reintegrated to create specialized healthcare operating system 50.

In yet a further embodiment, a software package is installed on an existing operating system. This software package adds all of the additional components required to turn the existing operating system into specialized healthcare operating system 50 without changing the hosting operating system. In this embodiment, an executable from the package will simulate health-specific GUI 80 and will mask the native GUI 80 of the hosting operating system.

Additionally, different embodiments of specialized healthcare operating system 50 may be customized for different domains of healthcare management.

In a preferred embodiment, specialized healthcare operating system 50 operates on a standalone personal computer hardware platform and can operate the standalone personal computer hardware platform without any medical device 20 installed. In other embodiments, specialized healthcare operating system 50 may provided for networked healthcare management systems, where health data are created and collected in multiple points such as over a local area network.

Specialized healthcare operating system 50 employs an enhanced dynamic and interactive healthcare specific graphical user interface, GUI 80. The health-specific GUI 80 is healthcare oriented and embodies a new concept in developing dynamic and interactive personally customizable user interfaces which permits taking in information without reading and actively supports a set of predetermined healthcare management concepts by itself.

Healthcare specific GUI 80 employs a unique and proprietary technique of arranging icons and menus in a display area such as display 8 (FIG. 1) and comprises health objects (not shown in the figures), i.e. healthcare-specific basic components based on healthcare related concepts. Healthcare specific GUI 80 dynamically changes and orders the display of icons, health objects, and menus according to a personal health condition and schedule, presents a comprehensive healthcare information portal, presents a variety of disease specific healthcare applications, forms a visual and personal healthcare portal, and interactively guides patients 1 through a healthcare portal.

The collected predetermined set of healthcare related functions, e.g. 54, are processed into an easy-to-access and user friendly GUI 80 graphical display. Patient 1 may then use GUI 80 to find those selectable items needed in a single visual display of displayed items comprising menus, health objects, icons, and options. The displayed items may comprise options for healthcare related communications, e.g. create a buddy list based on personal medical contacts list; healthcare information and news; healthcare management and treatment applications and healthcare directory services; drug information; and the like. The items may thus be presented in an integrated environment.

GUI 80 may further change and show different types of icons and health objects according to different diseases and/or other physical conditions of patient 1. The icons and health objects may change in color, shape, order, or other mutatable attribute according to different time schedules and severity or degree of some of findings, e.g. the shape, size, color, and relative position of each icon can change according to a current health status and health plan of patient 1.

GUI 80 can also provide a conventional personal computer environment as needed, temporarily masking health specific GUI 80 behind a more conventional user interface. In a preferred embodiment, healthcare tasks and functions of specialized healthcare operating system 50 remain active and running while GUI 80 is in a conventional personal computer mode.

GUI 80 may further include basic components, e.g. My Prescriptions which allows viewing and manipulation of medication, My Personal Visits which allows viewing and manipulation of calendars, My Medical Contact which allows viewing and manipulation of contact information, an emergency phone dialer, My Healthfolio which allows viewing and manipulation of personal health data, My Schedule Today which also allows viewing and manipulation of calendars, and the like.

GUI 80 may additionally be equipped with speech synthesis and speech recognition technology to employ audio-visual interactions.

In a preferred embodiment, GUI 80 is interactive and dynamic, and may automatically detect changes in health status and health plan for patient 1 and automatically react and rearrange the available options to best suit the condition of patient 1.

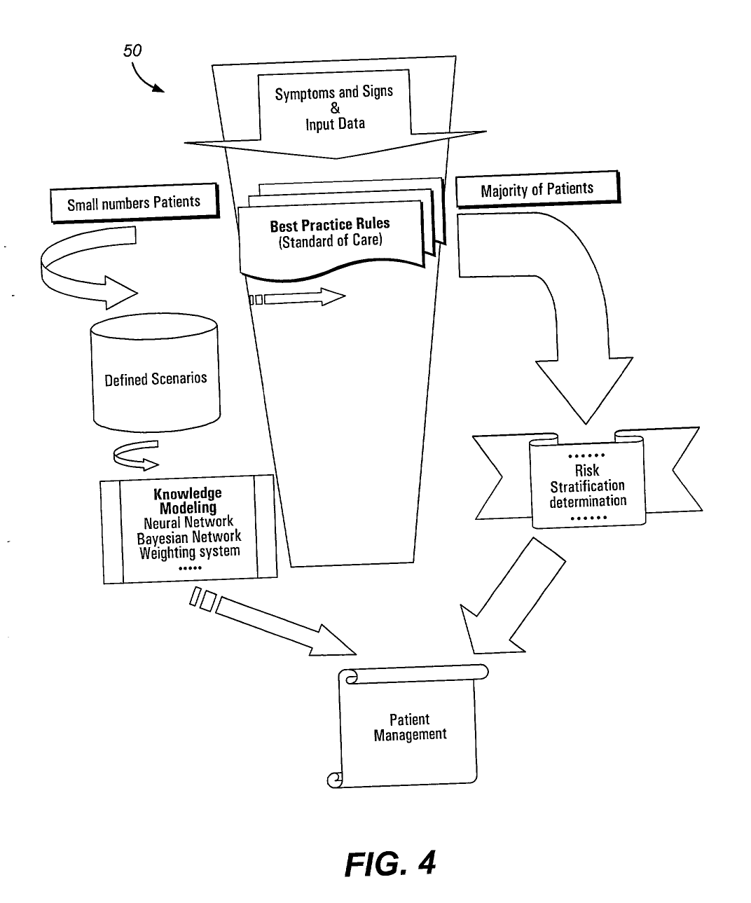

Referring now to FIG. 4, specialized healthcare operating system 50 possesses comprehensive disease based management protocols according to the best clinical practice algorithms and physician driven protocols. Specialized healthcare operating system 50 provides patients with virtual visits at home using management protocols. During a virtual visit, specialized healthcare operating system 50 starts a personally customized set of data acquisitions through medical devices 20 (FIG. 1) and a personalized complications, signs, and symptoms questionnaire to obtain the health data, e.g. hearthfolio 31 (FIG. 6). Specialized healthcare operating system 50 may compare acquired data against healthfolio 31 for patient 1 and simulate a healthcare data analysis using Physician Cognitive Engineering and/or Cardiology Cognitive Engineering such as would be experienced by patient 1 if patient 1 happened to be present in a best practicing healthcare facility.

Referring now to FIG. 5, specialized healthcare operating system 50 may provide patient 1 with sophisticated medical recommendations according management protocols and helps patient to act according the recommendations. Specialized healthcare operating system 50 may include a collection of disease specific or general healthcare management applications and patients can install other third party healthcare applications on specialized healthcare operating system 50.

Specialized healthcare operating system 50 may also be a platform for healthcare management applications provided by third party vendors. Using the pre-existing software components natively embedded inside specialized healthcare operating system 50, e.g. 54 (FIG. 3), developers can achieve standard medical data exchange, interoperability and communication with medical devices 20.

Referring now to FIG. 6, an exemplary interface to healthfolio 31, healthfolio 31 comprises an individualized, user specific health portfolio detailing information deemed relevant to a desired condition, e.g. heart health such as historical and current characteristics of patient 1. These data may be gathered and stored in a database for each patient 1, such as heartfolio database 30 resident in persistent data store 7. Healthfolio 31 may include an interface such as a displayable interface on output device 8 (FIG. 1) which permits gathering information relevant to previous and current heart and general health condition of patient 1. Healthfolio 31 information may be gathered during an enrollment of patient 1 prior to an acute cardiac event and, in a preferred embodiment, is stored in a relational, HIPAA compliant, secure database, e.g. heartfolio database 30. Additionally, healthfolio 31 information may be gathered during alternative means such as interactively over data network 100 to remote service 3, where remote service 3 may comprise a web page form, human operators at remote computer location 19 (FIG. 1), or the like or combinations thereof.

Heartfolio database 30 (not shown in the figures) comprises a database which stores and retrieves medical information for patient 1 which may include physiological data obtained through medical device 20 (FIG. 1), clinical information, family history, laboratory data, drug history, a base-line ECG and historical medical history for patient 1. These data may be used for analysis and future reference, and may be stored in a secure, HIPAA compliant relational database. The data may be made available through secure channels such as over data network 100 to physicians and healthcare providers for patient 1 for further reference and analysis.

Additionally, heartfolio database 30 may be used for further research, epidemiological surveys, and also future management of patient 1. For example, heartfolio database 30 may be used in part by personal healthcare management system 10 for early risk stratifying of chest pain for patient 1. Heartfolio database 30 may further comprise a plurality of measurements for patient 1, e.g. serial ECGs for patient 1 which may be used for risk stratification and diagnosis of the silent ischemia and myocardial infarction.

Heartfolio database 30 may also be used to maintain a transmittable data repository of actual base-line data that is not available in the hospital setting and may also accessible through secure data communication channels for use by physicians, health care providers and other users authorized by patient 1.

Referring now to FIG. 7, an exemplary interactive questionnaire, interactive questionnaire 40 is presented to patients 1, such as on output device 8 (FIG. 1), to obtain necessary information for instant risk stratifying of heart attack. Alternatively, interactive questionnaire 40 may be presented orally such as by remote service 3 (FIG. 1). Additionally, interactive questionnaire 40 may also be accessible through data network 100 (FIG. 1) such as by using a browser, touch tone telephone system, instant messaging, and the like, or combination thereof. In currently envisioned alternative embodiments, interactive questionnaire 40 may be integrated into a kiosk such as an automated teller machine (ATM) or may available to patient 1 by telephone such as via remote service 3 (FIG. 1).

Interactive questionnaire 40 may be based on physician designed interactive medical questionnaires for gathering symptoms and clinical information for patient 1. Patient 1 utilizes interactive questionnaire 40 to provide information concerning major signs and symptoms, current and past medical information, and other pertinent medical data for patient 1. In a preferred embodiment, interactive questionnaire 40 is interactive. Based on answers provided by patient 1, personal healthcare management system 10 can use interactive questionnaire 40 to provide targeted relevant questions for acquiring additional useful clinical data.

In a preferred embodiment, interactive questionnaire 40 utilizes a multi-media enriched interface which may include access via numerous functionally equivalent methods, e.g. mouse clicks, touch screen, and speech recognition technology.

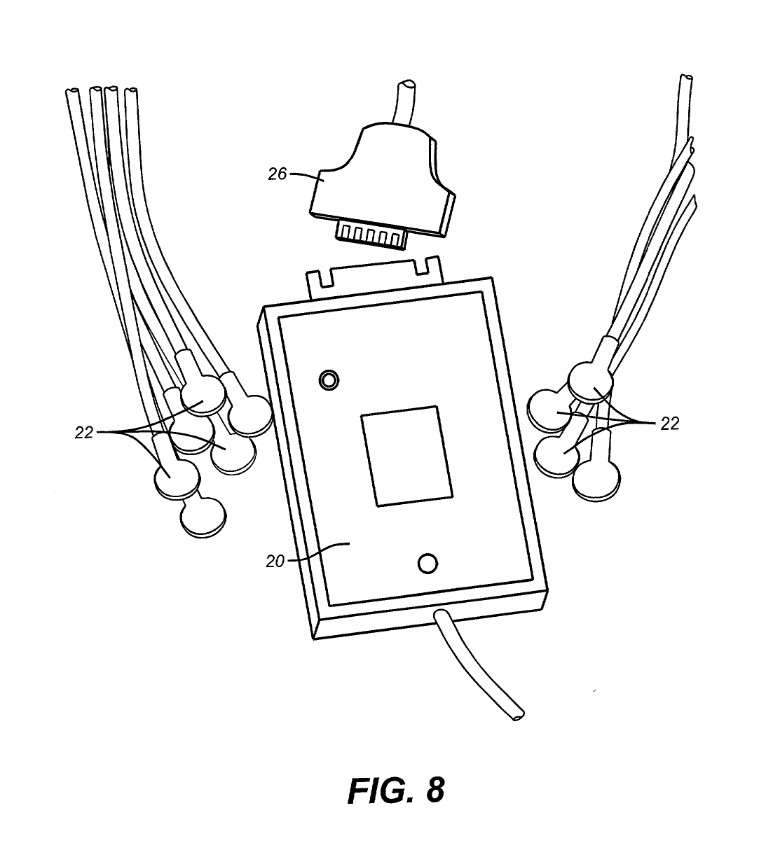

Referring now to FIG. 8, medical device 20 is operatively in communication with computer 11 (FIG. 1), such as via one or more serial, parallel, infrared, USB, or IEEE 1384 ports or the like or combinations thereof. Medical device 20 is used for acquisition of physiological data, e.g. ECG, heart and respiratory rate, blood pressure, and PO₂, from patient 1 (FIG. 1) and transferring data to personal healthcare management system 10 for managing patient 1. Medical device 20 may be a plurality of such devices.

Medical device 20 may be a commercially available or a proprietary device which, in a preferred embodiment, comprises a ECG system for acquiring heart signals. Additionally, medical device 20 may be external to computer 11 (FIG. 1) or internal to computer 11. Data representative of these signals may be integrated using an internal component of a computer 11 or provided via an external connection to computer 11. The external connection may be accomplished through any available data port or by a specially designed connecting device able to handle specialty devices such as medical devices 20.

As used herein, “medical device” may further comprise proprietary engine software 24 (not shown in the figures) and connecting media 26.

Proprietary engine software 24 enables acquisition, storage, processing, retrieval, and transferring of data to personal healthcare management system 10. Proprietary engine software 24 may compress the data for faster transportation of the data. Additionally, proprietary engine software 24 may be used to interpret data, analyze the input data, and provide output to patient 1, e.g. showing the results, giving feed back to patient 1, and providing medical instructions to patient 1. Proprietary engine software 24 may also provide personalized, individualized health management for patient 1 using computer 11.

Connecting media 26 connects medical device 20 to patient 1 to acquire physiological data from patient 1. Connecting media 56 may comprise a commercially available device, including electrodes, or a proprietary device such as a body wrap or article of clothing into which one or more sensors 22 and/or medical devices 22 have been embedded. Connecting media 26 may be used to send acquired physiological data through data network 100 (FIG. 1) in numerous functionally equivalent methods, e.g. wired or wireless methods, using numerous signaling protocols such as through the Internet, telephone, wireless media, satellite systems, and the like.

Referring generally to FIG. 9 and FIG. 10, embodiments of a integrating device 16, integrating device 16 comprises a portable device providing one or more data communication channels, generally referred to by the numeral “70,” which can accommodate digital and/or analog data generated by medical devices 20 (FIG. 1) to personal computer 11 (FIG. 1), data network 100 (FIG. 1), or a combination thereof.

Personal healthcare management system 10 may be designed and manufactured in variety of generic types and shapes to achieve portability, customizability, and affordability. In a preferred embodiment, computer 11 is made in one of several configurations, including desktop, a laptop computer, or a handheld device. In currently envisioned alternative embodiments, personal healthcare management system 10 may be built into a home appliances or furniture, e.g. part of a television or a couch. For example, a couch may combine personal healthcare management system 10 with a traditional couch to provide a combination of a full-featured personal computer 11, a plurality of medical devices 20, and a couch to further facilitate the use of variety of home based medical devices 20 along with a full-featured personal computer 11 at home. Similarly, another further embodiment of personal healthcare management system 10 can be integrated into a bed, e.g. a bed with a specialized built-in personal healthcare management system 10 and variety of bedside monitoring devices 20 including heart and respiratory monitoring devices 20 for disabled or debilitated patients 1 or patients 1 needing medical supervision.

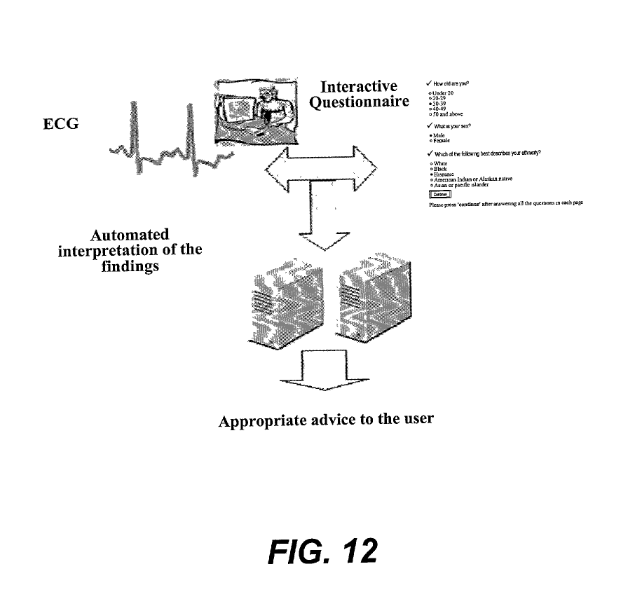

In the operation of an exemplary embodiment, referring generally to FIG. 11, medical diagnosis is accomplished by presenting interactive questionnaire 40 either on computer 11 over through an interactive system such as over the phone to remote service 3 (FIG. 1) to patient 1 (FIG. 1) during an initial period. Using input from patient 1 in response to interactive questionnaire 40, personal healthcare management system 10 creates healthfolio 31 (FIG. 6) and stores healthfolio 31 in heartfolio database 30. As needed, personal healthcare management system 10 obtains one or more physiologic measurements of a predetermined condition of patient 1 by using medical device 20. These are analyzed by medical diagnostic software 60 which may use data resident in heartfolio database 30 in making its diagnostic analysis. Once the analysis is completed, medical diagnostic software 60 presents its advice to patient 1 (e.g., FIG. 12). Further, medical diagnostic software 60 may invoke additional actions in response to its analysis, including calling emergency services.

Using its simulated expert physician reasoning, medical diagnostic software 60 compares the total risk of patient 1 with a defined risk for scenarios and cases which have been already defined and categorizes patient 1 based on this comparison. Medical diagnostic software 60 can be further self trained, e.g. use heuristics, based on available data in its database. The cases, their diagnosis, and their stratifications constitute a data set which is used for training system for risk stratification of patient 1 which can not be handled based on the best practice rules and algorithms and defined scenarios (FIG. 4).

Personal healthcare management system 10 may use multiple diagnostic and computational models to replicate the diagnostic process in hospitals. Using information input by patient 1, output from interactive questionnaire 40 is made available to medical diagnostic software 60 which processes the data to help quantify the risk of and initiate care for patient 1, e.g. cardiac risk and care. Medical diagnostic software 60 is also useful for risk stratifying of patient 1 based on current signs and symptoms, physiologic data, and data in heartfolio database 30.

In a preferred embodiment, medical diagnostic software 60 is based on physician cognitive engineering to simulate expert physician reasoning in field of cardiology and emergency medicine for risk stratification of patient 1 suspected of having heart attack and providing early management and decision support. Expert physicians may be studied to obtain detailed insights into the basic nature of clinical problem solving that a cardiologist or emergency physician usually deals with during evaluation of patient 1 suspecting of having heart attack. An evidence based medical approach using best clinical practice rules, guidelines, and clinical algorithms is then converted into a computational model for programming medical diagnostic software 60.

Cardiologists, emergency medicine physicians, and internists may further be presented with specially designed software which simulates a variety of scenarios and cases based on combinations of symptoms and physiological data. The diagnosis and risk stratifications of physicians may be then be stored in a database for each set of input clinical and physiological data and used as a base for a variety of risk stratification scenarios.

For atypical presentations which can not be risk stratified or based on best practice rules and algorithms driven by physician cognitive engineering and which are not compatible with the defined scenarios, personal healthcare management system 10 may use a knowledge modeling system, e.g. neural network, Bayesian networks, expert systems, and the like, or combinations thereof, to generate an appropriate risk stratification model.

In an embodiment, a knowledge modeling system for medical diagnostic software 60 comprises a cardiac risk weighting system which allows comparisons of a new case with one of previous defined scenarios. The knowledge modeling system extracts differences between the new case and the defined scenarios and assigns each new symptom and/or sign with a predetermined risk weight.

Medical diagnostic software 60 is able to interpret the physiological input data (ECG, BP, etc) from patient 1 and analyze it for risk stratifying of patient 1. In an embodiment for cardiac patients 1, the most important capability of personal healthcare management system 10 is its ability to interpret ECG signals. The results of this analysis is used for cardiac risk stratification of patient 1. For example, medical diagnostic software 60 may combine interpreted results of the physiological data of patient 1 (ECG, BP, etc) with clinical data and symptoms for patient 1 and uses its logic for risk stratifying and managing patient 1. Medical diagnostic software 60 categorizes patient 1 according to the risk of ischemic heart disease for patient 1 according to a plurality of categories, for example:

high risk, i.e. patients 1 whose risk of having ischemic heart diseases is greater than a predefined threshold, e.g. more than 7%, according to the risk stratifying system. Personal healthcare management system 10 may advise the high risk patient 1 to seek immediate emergency medical help as well as outline an away from hospital plan for that high risk patient 1, e.g. aspirin, call 911, cardio-pulmonary resuscitation, and the like. Additionally, medical diagnostic software 60 can automatically contact the local EMS service, activate an alarm system which is connected to the health care system of patent 12, or contact the health care provider hospital for patient 1.

low risk: patients 1 whose risk of having ischemic heart disease is low, i.e. below a predefined threshold, e.g. between 2-7%, according to the risk stratifying system. Personal healthcare management system 10 may advise the low risk patient 1 to come back to personal healthcare management system 10 for reevaluation after a predetermined interval, simulating a hospital observation center. If after reevaluation patient 1 falls into a high risk category, patient 1 may be informed of their changing condition with recommendations to go to the hospital and seek medical advice. Reevaluation of patient 1 can be repeated several times. Additionally, patient 1 can have a profile of previous physiological and clinical data like serial ECGs stored in heartfolio database 30 which can be used by their health care provider. Serial ECG and follow-up of the symptoms of patient 1 ensures that most of the ischemic events would be diagnosed by personal healthcare management system 10.

very low risk: patients 1 whose risk of having ischemic heart disease is below a predefined threshold, e.g. less than 2%, according to the risk stratifying system. Based on their current symptoms, their physiological data (ECG), and base line data from their heartfolio database 30, very low risk patients may be advised to make a non-emergency appointment with their primary care physician for evaluation of their symptoms. An advantage of personal healthcare management system 10 allows patients 1 to come back and be reevaluated whenever they had a new symptoms or they symptoms become worse.

It will be understood that various changes in the details, materials, and arrangements of the parts which have been described and illustrated above in order to explain the nature of this invention may be made by those skilled in the art without departing from the principle and scope of the invention as recited in the following claims.

What is claimed is:

1. A system for early risk assessment of a patient experiencing chest discomfort associated with a potentially emergency heart condition, comprising:
a. a computer comprising a persistent data store, an input device, and an output device;
b. a data network operatively in communication with the computer;
c. a heartfolio database resident in the persistent data store, comprising a healthfolio, the healthfolio further comprising an individualized, heart health portfolio for the patient;
d. an interactive questionnaire displayable on the output device, the interactive questionnaire able to interactively accept input from the patient relating to current symptoms and clinical information of the patient via the input device, the interactive questionnaire further able to store the input in the heartfolio database;
e. a medical device useful to acquire predetermined physiological data of the patient, the medical apparatus operatively in communication with the computer; and
f. medical diagnostic software executing at least partially in the computer, the intelligent software capable of assessing a risk of a predetermined health condition of the patient using the predetermined physiological data and data in the heartfolio database.

2. A method of medical diagnosis, comprising:
a. presenting an interactive questionnaire on a computer to a patient, the computer located at a residence of the patient;
b. using input from the patient in response to the interactive questionnaire to create a healthfolio;
c. storing the healthfolio in a heartfolio database;
d. obtaining a physiologic measurement of a predetermined condition of the patient using a medical apparatus;
e. analyzing the physiologic measurement using the healthfolio by medical diagnostic software; and
f. presenting advise to the patient based on the analysis.
